(19) United States
(12) Patent Application Publication
(10) Pub. No.: US 2024/0047019 A1
(43) Pub. Date: Feb. 8, 2024

(54) MODEL-FREE TIME-INVARIANT EVALUATION OF TRANSPORT AND EFFICACY OF CHEMICALS AND DRUGS

(71) Applicant: Seqvera Ltd. Oy, Helsinki (FI)

(72) Inventors: Michael Gasik, Helsinki (FI); Yevgen Bilotsky, Melbourne (AU)

(21) Appl. No.: 17/875,843

(22) Filed: Jul. 28, 2022

(51) Int. Cl. G16C 20/50; G16C 20/30; G16C 20/70

(52) U.S. Cl. CPC G16C 20/50; G16C 20/30; G16C 20/70

(57) ABSTRACT

The invention discloses a method for testing of transport and/or efficacy of chemicals and drugs by assessment of plurality of time-invariant parameters in a relevant environment, leading to higher confidence, predictive capacity and better risks assessment. The method makes use of pre-selected models obsolete as the combination of invariants is sufficient to understand the pharmacokinetics in many cases to predict the drug behavior. With this, the amount of in vivo or clinical tests required could be reduced with substantial savings of time and efforts. It is of a particular importance for the cases where such clinical tests are impossible or unethical to be performed, like on pregnant women due to risks to maternal and fetal health. Also, optimization of drugs design and their distribution satisfying biomedical requirements will be achieved with less time and costs, without explicit knowledge of its pharmacokinetics, mode or mechanism of action.

### PRIORITY

This application does not claim priority of any other applications.

### FIELD OF THE INVENTION

The present invention relates to a new method of testing of transport properties (and related efficacy) of chemicals and drugs from the source compartment to the target compartment, allowing model-free evaluation of plurality of time-invariant parameters describing the intrinsic transport behavior, performance or efficacy of that chemical or drug at proper conditions, potentially allowing prediction of that drug or compound behavior.

### BACKGROUND OF THE INVENTION

Transport of a chemical or drug from the origin of input (source) to the intended destination (target) is a key for any chemical or pharmacological process. Ideally the solid knowledge of the transport equation allows reliable calculation of that substance concentration in time which is critical to define e.g. a therapeutic or toxic effect of that compound. Correct and detailed drugs testing is rather time-consuming, and it requires proper experimental arrangements to establish a reliable link between in vitro and in vivo responses, known as IVIVC (in vitro—in vivo correlation).

IVIVC is a fundamental part of the drug discovery and development process, and it is usually expressed in a form of different models. These models should accurately predict the in vivo behavior of drugs based on in vitro observations, and their applications include pharmacokinetic properties testing, quality assurance, pharmaco-surveillance and quality control during the development and scale up of a formulation [1]. According to this reference, IVIVC is defined by the US Food and Drug Administration (FDA), as “a predictive mathematical model describing the relationship between an in vitro property of a dosage form and an in vivo response”. The property of solid dosage forms most commonly applied for in vitro development and quality control tests is the rate of dissolution of the drug and it is usually correlated with the concentration or amount of drug absorbed that reach the plasma circulation at certain time [1]. Therefore, a good IVIVC model based on dissolution tests should allow prediction of the in vivo behavior of drugs.

Whereas the correlation between in vitro dissolution or drug elution and in vivo absorption has been recognized, comprehensive models that include complementary properties to improve the predictive capacity still have to be developed. They are required taking into account the physical, chemical and biological factors involve in the dissolution and absorption of drugs [1]. The most common approach to in vivo pharmacokinetic and pharmacodynamic analyses involves sequential analysis of the blood plasma concentration- and response-time data to construct a kinetic model as an independent function [2].

The effect of a drug is determined by the amount of active drug present in the body particularly at the target site which depends on absorption, distribution, metabolism, and excretion (ADME) of that compound [3,4]. Most drugs are administered orally and need to be absorbed in the gastrointestinal tract to enter the bloodstream, allowing them to be transported to their site of action. On its way to the target site, the drug reaches the liver, where first-pass metabolism takes place and therefore the drug concentration—and thus its bioavailability—is reduced before entering systemic circulation. Intravenous drug administration bypasses the first-pass effect, resulting in greater bioavailability [4].

Pattern recognition in pharmacodynamic analyses contrasts with pharmacokinetic analyses with respect to time course [3] as the time course of drug in plasma usually differs markedly from the time course of the biomarker response due to numerous interactions (transport to biophase, binding to target, activation of target and downstream mediators, physiological response, cascade and amplification of biosignals, homeostatic feedback) between the events of exposure to test compound and the occurrence of the biomarker response [3].

Conventional analysis of such data, whether or not combined with the pattern recognition, usually comprises an overview of the curve (pattern), selection of the likely model or models which could be a set of algebraic and/or differential/integral equations, followed by the regression methods to find appropriate constants and finally trying to assess their meaning to estimate a predictive behavior and/or potential mechanism (pharmacokinetics and pharmacodynamics) [3,4]. The quality of information expected by the user of such methods should be not only sufficiently rigorous to provide scientifically based evidence on the material or tissue, but also to provide acceptable correlations, trends and predictions which can be safely used in design, development and applications of drugs and chemical substances in general. Hence, if the pattern was improperly recognized or the model selected was not the right one, wrong predictions can be made. Simple or more sophisticated regressive methods can result in computer-driven “best fit” equations which only differ from other alternatives by a minute fraction of decimals.

This problem has been addressed in numerous books, studies and publications. For example, in work [5] to describe kinetics of an ATR inhibitor, a set of 12 differential and 2 algebraic equations was required to be constructed, having 16 unknown parameters to be fitted. For identifiability, these parameters estimation and fitting were performed from 3,000 randomly generated starting values from the primary estimated values, with uniform sampling (in log space) between upper and lower set bounds. However, the goodness of fit was assessed using just the coefficient of determination, without other details for validity [5]. The need to estimate more than a dozen of unknown parameters by statistical fitting puts a strong requirement for the initial data quality. Noise, leverage or outlier points can have a huge effect of derivative of the signal and the quality of the outcomes could easily be compromised. Such data assessment is possible to carry out only when the drug effects and mechanism of actions by phases are known or could be reasonably assumed, but this does not guarantee that better values could be obtained with more or less composite differential or algebraic equations in any other combination.

In an example disclosed in Chinese patent application CN109753764, a two-dimensional simplified model for researching the drug sustained release process of the drug eluting stent is established, a straight or bent rectangle is used for representing a blood vessel domain, and N=1 . . . 20 squares or circles or polygons are used for representing a drug-loading coating stent; and establishing an approximate function relationship among the effective drug release time. There the combinations of simple diffusion differential equations are deployed where parameters of the coating and the stent are sought by adopting a Kriging agent model optimization algorithm, balancing local and global searches by using an expectation function, and stopping optimization when the optimization process meets a set convergence condition. According to this method, parameter optimization of the drug eluting stent is carried out through a finite element simulation and agent model optimization method, which is rather cumbersome and fits only this selected drug source form (stent in this case). No invariant kinetic or transport parameters are possible to obtain with this method.

In another example shown in Chinese patent application CN112730278, a mathematical modeling method for drug sustained release of tea polyphenol drug-loaded microspheres is disclosed. This method comprises the following steps: establishing a tea polyphenol standard surface equation; converting the formula to obtain a released drug concentration equation; performing conversion to obtain a release amount equation in the time period T; and dividing the time T into enough equal-length micro time periods to obtain a cumulative release amount equation, a dosing amount equation and an encapsulation rate. According to this method, drug sustained release behaviors of a tea polyphenol drug-loaded sustained and controlled release system are modeled again iteratively until a more accurate calculation model is explored. In practice, this example sets a pre-defined linear model linking analyte adsorption rate A with concentration x and time t in a form of λ=ax+bt+c and use of a curve fit software to carry out multiple regression processing to this array to obtain the value of coefficient a, b, c. This is the standard mathematical approach of the linear regression which does not reveal any invariant parameters, but it does require linearity by default and leads to the fitting statistical coefficients values with no physical meaning. This method cannot be used for those chemicals or drugs which do not have a proper analyte adsorption rate.

In yet another example of US patent U.S. Pat. No. 7,286,970, a method for tailoring multidrug chemotherapy treatment regimens to individual patients is disclosed, comprising the steps of: providing a set of differential equations representing a mathematical model of rates of population change of proliferating and quiescent diseased cells, said mathematical model including parameters corresponding to cell kinetics and evolution of drug resistance of the diseased cells to cell-cycle phase-specific cytotoxic drugs, cell-cycle phase non-specific cytotoxic drugs, and cytostatic drugs; obtaining cell kinetic parameter values from an individual patient including proliferative index, apoptotic index, cell cycle time, and level of drug resistance; using the cell kinetic parameter values obtained from the patient to solve the set of differential equations of the mathematical model to determine a plurality of treatment regimens for the patient, each of said treatment regimens having a quantitative efficacy value associated therewith representing the efficacy thereof in reducing the diseased cell population of the patient; and selecting one of the treatment regimens having an efficacy value that is desirable for treating the patient, said selected treatment regimen thereby being tailored to the patient.

This method is realized in a form of a computer program which solves a set of pre-defined differential equations, and where these equations are based on pre-defined models such as a Gompertzian curve for modeling tumor growth and complex differential equations (as depicted in the Appendix D of the US patent U.S. Pat. No. 7,286,970). In fact, this method needs inputs of cell kinetic data obtained from an individual patient and not obtained during the tests themselves. As patient data are required, testing of a drug in vitro without these data is not feasible according to this patent method. As in previous examples, parameters and coefficients for these differential equations are needed to be guessed or obtained with iterative fitting of the data, becoming ad hoc values for a specific case.

All these methods have systemic limitations comprising at least one of the following: a need of pre-defined or pre-selected model, a set of pre-formulated differential equations, a guess or a knowledge of the potential drug acting mechanism, an application of the regression to the data (without explicit check whether or not such methods are deployable), a need of determination (guess and/or iteration) of number of fitting parameters or numerical series, absence of the invariant parameters which have a clear physical meaning, and the lack of transport kinetic equation in a closed analytical form, which potentially could enable the prediction.

The drawbacks of such simplified methods have been recognized in the literature stating the fact that chemicals and drugs transport processes, especially in living systems, are usually far too sophisticated to be expressed or fitted with a set of simple algebraic or standard partial differential equations. Even if experimental data could have been reasonably fitted, the number of unknowns and computational efforts required would make such operation obsolete for practical use.

In example presented by Popovic et al. [6], a classical analysis widely used to predict time evolution of a drug concentration for different types of drug introduction is criticized for the drawbacks shown above, and for the lack of justification of hypotheses required to prove the applicability of it for pharmacology analyses. Common compartmental models of pharmacokinetics are, in general, mathematically formulated as systems of ordinary differential equations with specified initial and boundary conditions. In work [6] use of fractional calculus is suggested as it is able to catch the “memory effect” of the systems in question. The integer order derivatives, commonly used in the kinetic equations, take into account only local properties of functions (at time t), while fractional derivatives take into account all values of functions in a time interval [0,t] in which a process that is analyzed takes place hence taking a history of a process into account [6].

The method used in publication [6] relies on fractional order derivatives with a multi-compartmental model using Laplace transform, which is not a common operation a lab technician usually able to perform. The method was demonstrated in [6] on two experiments were performed with testing 12 healthy volunteers with a slow release 100 mg diclofenac tablet formulations (=the source compartment in the terminology of the recent invention). The resulting simplified equation for time changes of the concentration in the blood plasma (=the target compartment in the terminology of the recent invention), designated as c₂(t) in [6], was expressed with six unknown parameters (d, k₀₂, k₂₁, α₁, α₂ and the lag time) as:

$${c}_{2} ( t ) = d \cdot {\int }_{0}^{t} [ \frac{{E}_{{α}_{2} , {α}_{2}} [ - {{k}_{02} ( t - z )}^{{α}_{2}} ]}{{( t - z )}^{1 - {α}_{2}}} {E}_{{α}_{1}} [ - {k}_{21} {z}^{{α}_{1}} ] ] dz \qquad ( 1 )$$

where functions

$${E}_{α} ( y ) = {\sum }_{n = 0}^{∞} \frac{{y}^{n}}{Γ ( n α + 1 )} ; {E}_{α , β} ( y ) = {\sum }_{n = 0}^{∞} \frac{{y}^{n}}{Γ ( n α + β )} \qquad ( 2 )$$

are known as Mittag-Leffler functions [7] with ‘y’ being in general a complex number y=a±b·i; i=\sqrt{−1}. Unknown parameters were found by the least-squares fitting method and with the particle swarm optimization numerical procedure with the Matlab software [6]. This procedure is rather cumbersome, and the convergence of the numerical iteration for Mittag-Leffler functions (2) is not always guaranteed—for example, it also may take negative values for some combination of parameters [7] which does not have sense for a physical property like concentration. The variation of the parameters found in [6] was large (d=22.79±16.41 and k₀₂=1.51±1.05), making practical application of method (1) in clinical practice very difficult. Equation (1) in general is not even possible to calculate analytically in a closed form, and it is also impossible to expand it to more sophisticated multipart systems with growing numbers of unknown parameters.

Despite some advantages achieved in work [6], the constants obtained through the regressive fitting of the data are still based on the adopted models of the drug transport, with the main difference from classical analysis that ordinary differential equations were replaced with fractional differential equations. This approach is known for years in general pharmacokinetic analysis [6,7] but due to sophisticated numerical computations required it did not find practical applications. Verotta [7] has stated that the bottlenecks of this kind of analysis are in the stable evaluation of Mittag—Leffler functions (2) and their derivatives which is a non-trivial task, and in the lack of routines to evaluate the convolution of Mittag-Leffler functions (2) with typical drug inputs. In total, this method makes regular data fitting even more cumbersome and it is one of the reasons why it is seldomly used.

None of known or above presented methods is capable to evaluate model-free time-invariant parameters of the chemicals transport simultaneously in one single test from a single specimen. There is no known method able to get simultaneously a set of time-invariant parameters including transport constant, equilibrium coefficient of partition, characteristic times, kinetic parameter (value), volume of distribution, etc. without pre-selection of some mathematical model and without use of differential kinetic equations.

### SUMMARY OF THE INVENTION

Accordingly, this invention provides solutions that none of the known disclosures are able to provide.

This invention addresses testing of chemicals and drugs transport kinetics from the source compartment to the target compartment, aiming on experimental extraction for plurality of their properties, especially where these properties are functions depended on testing and environmental conditions, in the most cases, in an unknown way.

Furthermore, the invention also addresses obtaining a plurality of time-invariant parameters simultaneously without application of assumed or pre-selected models and without assumption of the data linearity. In addition, the objective of the invention is to use these properties to compare and assess performance of chemicals and drugs where these invariant properties are intrinsic to the nature of the system those chemicals or drugs are being deployed into.

The inventors have experimentally discovered that a properly carried test for transport of the chemicals and drugs specimen with measurement of at least concentration of the chemical or drug in one of the compartments in time alone, can be used to evaluate true time-invariant transport obtained with time convolution with idempotent analysis, without use of presumed materials models (zero-order, n-order etc. kinetics, or more complicated differential equations), and without need of complex transforms or functions like Mittag-Leffler ones.

The inventors have also experimentally discovered a way of linking these time-invariant properties directly relating them to expected chemical or drug transport behavior, connected with their biological activity and possible clinical actions. This would enable to achieve test results capable to answer whether a selected chemical or drug dose and means of deployment are better or worse for its intended application vs. other known or control substance, how different chemicals might affect each other transport and expected clinical performance, and how much time is needed to reach or not to reach the expected concentration of the chemical or drug in a predictive way.

According to the present invention, a method for determining transport capability and/or related efficacy of a chemical or a drug to transport in designated conditions is provided. The method of data analysis employed in the present invention do not require any prior knowledge about the chemical or drug properties, structure, mechanism of action or other behavior.

The test method comprises at least the following steps: placement of a drug or chemical specimen in a source compartment, establishing of a contact of that specimen with the transfer media (such as a fluid), periodic measuring of the changes in the concentration of the chemical or drug at least in one compartment as function of time and applied conditions, processing these history-dependent measured data by time convolution without application of a material transport (kinetic) model, calculation of the time-invariant transport properties from these measured data, and optionally comparing the results with the reference or control specimen.

The test method provides means to answer/evaluate whether a selected chemical or drug dose and means of deployment are better or worse for its intended application vs. other known or control substance, how different chemicals might affect each other transport and expected clinical performance, and how much time is needed to reach or not to reach the expected concentration of the chemical or drug in a predictive way. Such evaluations allow for example determination of safe dosing range of a drug, determination of suitable population for use of a drug among others. Moreover, such evaluations can be used in clinical trial in early determination whether a candidate compound should be tested further.

The test method may comprise inclusion of the other secondary conditions such as application of varying fluid composition, temperature, or presence of living media (such as cells or tissues) or be deployed directly to the clinical tests in animals and humans where only limited amount of information can be collected. The fluid media may comprise at least one of the following: water, saline or buffered solution, simulated body fluid, extracellular matrix liquid, blood or blood substitute, designated cells culture, bacteria culture, virus culture, pharmaceutical or biological compound or any combination thereof. The fluid might be additionally adjusted and monitored by its composition, pH, temperature, viscosity, pressure, or flow velocity, when it has relevance for intended application. Furthermore, the fluid can be also “virtual”—such as in the tests where it is not clearly known in which body fluids exactly this specific drug is being transported during the clinical trials.

An example of time-invariant functions is at least one of the following: transport constant, equilibrium coefficient of partition, characteristic time, kinetic value, volume of distribution. Similar properties linked with presence and concentration of other chemical or biological species, or any changes of any of the above, also as an efficacy parameter, can be also employed, either separately or in a combination.

One essential difference of the method of this invention vs. prior art is that mentioned time-invariant properties are calculated from the processed test data by time convolution without application of Fourier or Laplace transforms, use of conventional complex numbers algebra, an assumption of linearity of the functional properties, application of Mittag-Leffler functions, and without a need of pre-selection or assumption of the models for kinetics or transport.

In summary the invention discloses a method for testing of transport and/or efficacy of chemical compounds and drugs by assessment of plurality of time-invariant parameters in a relevant environment, leading to higher confidence, predictive capacity and better risks assessment. The method makes use of pre-selected models obsolete as the combination of invariants is sufficient to understand the pharmacokinetics in many cases to predict the drug behavior. With this, the amount of in vivo or clinical tests required could be reduced with substantial savings of time and efforts. It is of a particular importance for the cases where such clinical tests are impossible or unethical to be performed, like on pregnant women due to risks to maternal and fetal health. Also, optimization of drugs design and their distribution satisfying biomedical requirements will be achieved with less time and costs, without explicit knowledge of its pharmacokinetics, mode or mechanism of action.

### DESCRIPTION OF THE DRAWINGS

FIG. 1 presents the examples of the principle of the test method

FIG. 2 presents a flow chart showing the principle of the test data processing,

FIG. 3 presents given and calculated concentrations in the target compartment vs. time in example 1,

FIG. 4 presents the comparison of given and calculated concentrations for example 1,

FIG. 5 presents the comparison of given and calculated concentrations of diclofenac in clinical tests for example 2,

FIG. 6 presents the comparison of given and calculated concentrations of Claritin D release for example 3,

FIG. 7 presents the found clusters of formulations for kinetic value and characteristic time for example 4,

FIG. 8 presents the composite criterion for the best drug formulation selection for example 4,

FIG. 9 presents the discovered clusters for the formulations of example 4,

FIG. 10a, b presents a) the original data from the reference and b) the same data but analyzed in the present invention

FIG. 11 presents the changes of the instant partition coefficient with elution time for example 5,

FIG. 12 presents the changes of kinetic parameter with time for example 5,

FIG. 13 presents the comparison of measured and calculated concentrations of gentamicin release for example 5.

FIG. 14 a, b presents the comparison of caffeine concentration changes in the target as original data (a) and vs. generalized Deborah rate number (b) for example 7.

### DETAILED DESCRIPTION OF THE INVENTION

### Definitions

For the reasons of clarity, the following specific definitions are used in this invention:
“ [symbol] -value”, “kinetic parameter” or “kinetic value”—a time-invariant property of the transport process of the chemical or drug, having the positive value, representing the tendency of the material to delay, accelerate or lag its transport in the specific conditions. The higher is the value, the more non-linearity in time the transport process has. Beta-value is a constant when there are no changes in the process mechanism or kinetics as well as changes in the conditions such as in the volume of distribution (VOD). If there are changes in the conditions or nature of the transport process/system, then kinetic parameter values can vary over time approaching some equilibrium value.
“Characteristic time”—a time-invariant measure of the chemical or drug in the system analyzed representing the inertia of that system to reach a developed transport process. The higher is characteristic time, the longer it takes for the chemical or drug to reach a developed (normal) transport phase at other equal conditions. Characteristic time divided by the real experimental time is proportional to non-dimensional Deborah number (De).
“Coefficient of partition”—is expressed as the ratio of concentrations (in the simplest case) of the chemical or drug being analyzed in the source compartment to target compartment. Coefficient of partition can be constant indicating equilibrium distribution of the drug between source and target compartments, or it can change in time (when the experimental boundary conditions change in time) approaching the equilibrium value. In the present invention it is a time-invariant measure which is assessed experimentally.
“Comparison criterion”—is a real number or function, composed of the time-invariant properties. In the simple case it can be calculated as the characteristic time divided by the product of transport constant, kinetic parameter and coefficient of partition. It is used to select the best working pharmaceutic formulation among the list of options—for example, for the gastro-retentive dosage forms, this criterion is preferably to be the largest.
“Compartment”—a physical or virtual space where the chemical or the drug is initially introduced (source compartment) and where it is being transported to (target compartment). Compartments may have physical or virtual interface(s) between them. Examples of compartments might be (for an in vitro test) a tablet having the drug (=source) and a fluid volume where the drug is supposed to be eluted into (=target). For an in vivo test, an example could be a dose added (=source) which will enter into the blood system (=target). In the present invention it is not relevant how many intermediate compartments may exist between source and target compartments, neither whether there are losses in parallel.
Generalized “Deborah rate number”—a new non-dimensional parameter discovered by inventors which links real experimental time with the characteristic time and kinetic parameter. It might be considered as a reduced (normalized) time enabling consistent quantitative visualization and comparison between different data, as explained below in the examples.
“Efficacy parameter” or “Efficacy coefficient”—a surrogate convoluted parameter which is linking acting drug concentration in the target compartment (in this case usually unknown or unmeasurable) with a measurable physiological, biomechanical or biochemical readout which results as a consequence of that drug deployment. An example could be cardiac electrical current or blood pressure changes due to administration of the drug in question. This parameter is useful when there is a need to convert measurable value into unknown concentration.
“Idempotent analysis”—a method of mathematical analysis using operations substitution for linearization of a problem to be solved without alteration of initial variables, involving time convolution, observing causality principle (response always come after the stimulus applied), respecting the boundaries of thermodynamics (no violation of conservation laws), and accounting for non-local effects. It differs from conventional mathematical analysis, where the derivative of a function is always local.
“Time convolution”—a mathematical operation employing integration in time to obtain resulting average values of a property or a function.
“Time-invariant property”—a true real (not imaginary or complex) property (or function) of a specimen which may depend on other properties but does not depend explicitly on time or frequency of applied stimulus. Time-invariant property also includes specimen history data obtained by time convolution.
“Transport constant”—in the present invention is a non-dimensional, time-invariant measure which relates the capacity of the target compartment to accept the drug or chemical amount (or expressed as in concentration) at the test conditions. The higher is the value of the transport constant, the less drug is passing from the source to the target within equivalent time scales.
“Volume of distribution” (VOD)—a physical or virtual space volume of the respective compartments where drug or chemical are being introduced (source) or transported into (target). VOD can be unknown for some of the cases and can be in this case arbitrarily fixed, then VOD of another compartment is being estimated analytically.

Thanks to the employed method according to the present invention, a combined characterization of chemical or a drug transport is accomplished. The primary test method of this invention is an in vitro test, referring to a test performed outside a living body, but it can be deployed for in vivo test data analysis as well as for the analysis of the readouts from the clinical trials where sufficient data are available. The test method of this invention comprises at least the following steps:
a) placing a defined amount of a chemical compound specimen in a source compartment;
b) establishing or ensuring a contact of the specimen with a transfer media which is able to transport the compound to at least one target compartment;
c) measuring the concentration of that chemical compound or its derivatives, or at least one efficacy coefficient, in at least one of the compartments;
d) processing the measured data by time convolution procedure in real numbers without application of a preselected kinetic model;
e) calculating time-invariant parameters comprising a set including at least a transport constant, coefficient of partition, kinetic parameter and optionally efficacy coefficient, from the processed data;
f) repeating steps c)-e) until desired time of the experiment is reached;
g) generating the model-free equation for the compound transport between the compartments;
h) calculating the non-dimensional Deborah rate number for the data from steps c)-g);
i) optionally calculating the comparison criterion between the specimens or with the reference or control specimen.

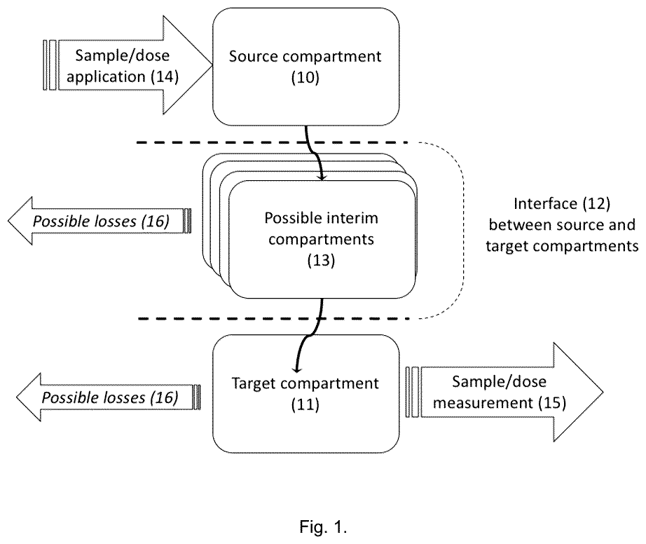

Referring to FIG. 1, in some example embodiments the method comprises application of a specified amount (dose or concentration) 14 to the first (source) compartment 10 (can be for example a tablet, a syringe, a specified concentration already dissolved, etc.). The resulting concentration of the chemical or drug 15 is measured from the second (target) compartment 11. There also efficacy parameter(s) can be used as surrogate readouts for 15. The method acknowledges that the interface 12 between compartments 10 and 11 can be a single interface, barrier or it can have unknown number of interim compartments 13. It is possible that some part of the chemical or drug tested can be lost 16 in these interim or target compartments 13 such as due to ADME, but for the present invention it is not essential, as only those readouts 15, which can be measured, have sense. The losses 16 if present will be incorporated with the time convolution procedure as embedded into the invariant parameters.

The readouts 15 may be measured or monitored simultaneously or off-line with any known and feasible physical or chemical method, providing that such measurements would not cause a significant or uncontrolled perturbation of the whole system of compartments, whether in vitro or in vivo. It is evident for one skilled in the art that such test arrangement could be implemented in different ways.

The method also provides enhancement of the efficient evaluation of a chemical or drug transport thanks to time-invariant parameters in relevant environment with a higher confidence and predictive capacity as well as risks assessment. With this, the amount of in vivo or clinical tests required could be reduced with substantial savings in time and efforts, and also optimization of drugs design and their distribution satisfying biomedical requirements will be achieved with less time and costs.

According to an embodiment, the method provides a combined characterization, i.e. simultaneous measurement and calculation of plurality of parameters, required to get an answer how the material tested fits into its intended application and whether it is better or worse versus control or reference. It is of a particular importance for the cases where such clinical tests are impossible or unethical to be performed, like on pregnant women due to risks to maternal and fetal health.

The key element of the data processing is based on time convolution and non-local, causal idempotent analysis. As shown above this approach is completely different from commonly used pharmacokinetic laws and models [1-7] and complex algebra. For biological systems one often cannot set up experiments to measure all of the state variables. If only a subset of the state variables can be measured, it is possible that some of the system parameters cannot influence the measured state variables or that they do so in combinations not defining the parameters' effects separately. It is well known that in general case such parameters are unidentifiable and are theoretically inestimable. Therefore, a common solution is normally to pre-select a model of the system, to guess initial estimates of the values of all parameters and conduct experimental data analysis using that model [1-7]. The present method does not need such operations. The new method uses integration with time convolution (global operation) instead of traditional differentiation (local operation), which stabilizes the calculation process and the output.

In brief, the data obtained are digitized, recorded or stored in a form of computer file or as a part of a database. It is essential that analysis according to the present invention should be carried during the experiment (or when carried after, all intermediate time points are to be known)—not like in the U.S. patent Ser. No. 10/379,106, because time convolution procedure in the present invention is based on the different approach shown below.

The data analysis background of the invention is as follows. Experimental data 14 and 15 (FIG. 1) are always functions of time but time is not a true coordinate in real life as it is impossible to move back and forth in time in the same way as for a spatial coordinate. Furthermore, complex physical-chemical quantities like in method (1)-(2) with Mittag-Leffler functions, do not exist in real world—it is impossible to have e.g. a mass like 4.2+0.6·i kg. All measurable physical and chemical quantities are described by real numbers. Hence, complex transforms alike Fourier transform over real physical quantities could not be considered feasible in practical deployment of any such solution (beyond just in abstract mathematical operations).

From the time convolution between two measurements t₁ and t₂ the change in the concentration c₂(t) in the target compartment in this invention is described by:

$${c}_{2} ( {t}_{2} ) = {c}_{2} ( {t}_{1} ) + \frac{1}{K \cdot Γ ( β )} {\int }_{{t}_{1}}^{{t}_{2}} \frac{S ( {t}_{1} ) dz}{{( {t}_{2} - z )}^{1 - β}} \qquad ( 3 )$$

where S(t) is the acting stimulus for the transport of any possible mechanism(s), K is the transport constant, [symbol] is the kinetic parameter, and ¬(·) is the gamma-function operator. The transport constant K and the kinetic parameter are two first principal invariants to be determined with the present analysis. In the simple case of two compartments (source 10 and target 11 in FIG. 1), with a single dose and the absence of the explicit sinks 16, the stimulus S(t) can be simplified to

$$S ( t ) = {c}_{1} ( 0 ) - {L}_{eq} {c}_{2} ( t ) ; {L}_{eq} = {lim}_{t \to max} \frac{{c}_{1} ( t )}{{c}_{2} ( t )} \qquad ( 4 )$$

where L_{eq} is the equilibrium partition coefficient (invariant parameter) of the drug or chemical in question between the compartments. Here the concentrations in the first (source) and second (target) compartments can be expressed as:

$${c}_{2} ( t ) = \frac{{m}_{2} ( t )}{{VOD}_{2} ( t )} ; {c}_{1} ( t ) = \frac{{m}_{0} - {m}_{2} ( t )}{{VOD}_{1} ( t )} \qquad ( 5 )$$

where m₀ is the e.g. drug mass is delivered to the source compartment at t=0, m₂(t) is the mass of the drug in the second compartment, VOD is the volume of distribution (can be constant or time-dependent). The expression (5) reflects the mass balance of the substance in question and can be complemented with additional items like sinks 16 in FIG. 1 or additional ADME related kinetic constrains where available.

By virtue of the dimensionality of the physical and chemical transport processes, and the fundamental idempotency causality principle (no response can be seen before the stimulus have been applied), there also another invariant parameter appearing after carrying out the procedure (3), namely the characteristic time τ of the process. It has a direct physical sense showing the inertia of the system to the stimulus. The ratio of this time to real experimental time can be seen as an equivalent to the dimensionless Deborah number (De) in rheology.

The inventors have experimentally found that a new dimensionless quantity which was named “generalized Deborah rate number” (Dr), is better capable for the representing of the integrated behavior of the system than De numbers. In the simplest case Dr= [symbol] where τ is the system characteristic time, t is the time of observation, and [symbol] (t)>0 is the kinetic parameter in (3), which also can be time-dependent. The latter reflects the changes of the transport process (acceleration or deceleration) with time.

The Dr number has a fundamental meaning for the test system as it corresponds to the reduced effective time of the transport process for this particular combination of the conditions. By plotting experimental data vs. Dr for different experiments, various time scales and conditions can be consistently visualized and compared.

The transport constant K reflects the ability of the chemical or drug in question to be distributed between target and source compartments—the higher is the value, the less drug can be delivered to the target. Together K, τ, [symbol] and L_{eq} constitute the coherent set of invariants capable to describe and even predict the behavior of the system. The remarkable feature is that equations (3-5) can be always numerically explicitly computed without need of assumptions of functions linearity or being of some specific type.

Depending of the mode of testing one or another set of data is retrieved, converted and processed with a computer algorithm. Many algorithms are known, however they are not suitable for the present invention, as they do not foresee extraction of the time-invariant parameters according to the present technique. The method of the time-invariant analysis previously invented in the patent U.S. Ser. No. 10/379,106, is not applicable to the present manner as it explicitly requires primary data from mechanical stimulation of the biomaterial (an implant or a scaffold, which could not be a drug or a chemical substance being analyzed in the same time) and the completion of the experiment (because the full history of the specimen should be embedded into the data). In that method any associated possible drug release measurements from a biomaterial specimen do require an explicit mechanical stimulation of that specimen so it cannot be treated with the equations (3)-(5).

The present method might be implemented in one or another dedicated computer code or software which specific precision, efficacy and processing time might be chosen depending on the problem addressed and number of the data points to be treated.

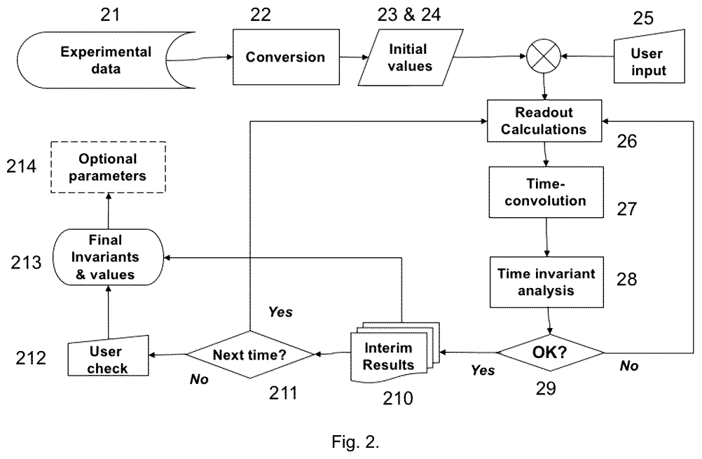

This new generic algorithm according to the present method is depicted in FIG. 2, and it includes as least:
retrieving the experimental data 21,
conversion of the data into real/virtual concentrations (22) at least for one of the compartments,
possibly setting initial mass of the sample with known or assumed VOD (23) and initial value of L_{eq} (24) by user interference 25,
calculating or locating concentrations (readouts) in the second compartment 26,
processing them by iterative time convolution 27,
executing time-invariant analysis 28,
checking quality and errors of the procedure 29 and re-iterating if necessary,
recording the interim results 210 into a computer file or database,
repeating the procedures 26-210 for the next time point,
finishing the procedure 211 when the last time point is processed,
analyzing by user 212 the whole obtained results set,
analyzing invariant parameters dependence on boundary and other conditions 213 and
optionally calculating 214 other parameters such as efficacy parameter or other criteria as required by a specific case.

Specific details of the algorithm and method of analysis are depending on the modality of the test and shown below in examples in more detail. For one skilled in the art it is also evident that some steps in the above procedure could be amended or skipped.

Another essential feature of the analysis according to FIG. 2 is the targeting on time-invariant properties determination rather than presentation of time-dependent data. Obtaining time-invariant properties is an important objective, as it would allow forecast of the specimen behavior in time beyond the limits of practical experiment.

Yet another feature of the analysis is the comparison of the time-invariant properties with other specimens or with the control (reference) specimen. This minimizes the risks caused by determination of absolute values at too different time scales. Whereas the comparison can be also carried out for any other measurements, here mapping the time-invariant property to another property or within the reduced time scale (generalized Dr numbers) may reveal hidden trends in behavior. Some of these trends are shown in the examples as discovered by the inventors experimentally.

For one skilled in the art it is also evident that this analysis can be combined with other measurements for example cytotoxicity or gene expression with live cells or tissue samples, or any relevant combination of the parameters of interest. There obtained invariants can be combined with other essential readouts and outcomes to enhance their value.

Advantages of the New Method

The present test method has essential differences from known pharmacokinetic models [1-7]. These differences and advantages are as follows.

First, the method according to the present invention does not stipulate that the chemical or drug transport has to be compliant with some pre-selected model (e.g. zero or fractional order kinetics, etc.), and does not need extra assumptions or measurements of the drug or chemical mechanisms of action. Selection of the model in any combination or fitting the data to one of such models, is de facto obligatory for any conventional calculations in pharmacokinetics.

Second, all invariants obtained in the present invention have a clear physical meaning and are real numbers which can be further used in the development or prediction.

Third, the invariants processed do not use complex algebra (such as Fourier or Laplace transform) neither sophisticated functions (such as Mittag-Leffler functions) for obtaining real properties. Instead, these data are being directly analyzed during the test by time-convolution and idempotent methods to result into the time-invariant properties, which are the true properties of the system in question, not linked to any theory or assumption.

Forth, analysis does not require that applied chemical stimulus S(t) signal have some specific form and thus can be applied to any arbitrary one in any sequence.

Fifth, the results of analysis are determined solely by experiments and do not rely on known, assumed or pre-selected models or requirements.

Sixth, the method has enabled a possibility in results comparison vs. new non-dimensional number (generalized Deborah rate) which in combination with efficacy parameter and the performance criterion can lead to a new way of screening or assessing of the activity or efficacy of different drugs or transport of different compounds in both laboratory conditions and clinical environments.

According to certain embodiments of the invention the following practical uses can be achieved. Detailed description of the experimental arrangements is provided in examples as indicated below:
Example 1 presents a mathematical simulation using known data which are well-defined in their statistical parameters. This demonstrates descriptive ability of the new method to extract important kinetics invariants not possible with common analysis.
Example 2 presents clinical data processing based on the drug pharmacokinetics measured and averaged for 12 patients. This example exhibits how data reported in literature can be re-processed with new method to extract important kinetics invariants without application of a pre-selected model.
Example 3 uses data from the test of a hydrophilic drug release, showing that experimental points could be processed with new method without a need of the model.
Example 4 processes the experimental drug release data in nine different formulations with fluctuating plasma levels and shows how this new method of the present invention allows model-free optimization of the drug delivery system.
Example 5 shows the proprietary experimental results in a very controlled conditions for the release of antibiotic formulation from coated samples. The present invention allows quantitative observation of the variations, extraction of the invariant parameters and unknown partition coefficient, without a need of assumptions.
Example 6 demonstrates extended features of the invention when the efficacy of the drug is being measured rather than its direct concentration change. This example shows how these data can be used to estimate the efficacy of the drug.
Example 7 demonstrates the unexpected features of caffeine transport kinetics via artificial simulated placental barrier in vitro which were discovered with the present invention method.

The objects of this invention include at least aspects of the following clauses:

1. A method for determining model-free time-invariant transport and/or efficacy properties of a chemical compound said method comprising the steps of:
a) placing a defined amount of a drug or chemical compound specimen in a source compartment;
b) establishing a contact of the specimen with a transfer media which is able to transport the compound to at least one target compartment;
c) measuring the concentration of the drug or chemical compound or its derivatives, or at least one efficacy coefficient, in at least one of the target compartments;
d) processing the measured data by time convolution procedure in real numbers without application of a preselected kinetic model;
e) calculation of time-invariant parameters comprising a set including at least a transport constant, coefficient of partition, kinetic parameter and optionally efficacy coefficient, from the processed data;
f) repeating steps c)-e) until desired time of the experiment is reached;
g) generating a model-free equation for the compound transport between the compartments;
h) calculating a non-dimensional Deborah rate number for the data from steps c)-g) representing the transport and/or efficacy properties; and
i) optionally calculating a comparison criterion between the specimens or with the reference or control specimen.

2. The method of clause 1, wherein data analysis is executed iteratively for discover unknown coefficient of partition of the compound between the compartments, and/or efficacy coefficient, and/or unknown volume of distribution.

3. The method of any of the previous clauses, where the data analysis and comparison between the experiments, control(s) and/reference(s) are being made versus non-dimensional Deborah rate number.

4. The method of any of the previous clauses, wherein the method comprising multiple experiments including control(s) and/reference(s), wherein at least one efficacy coefficient is also determined step c) for the multitude of experiments, control(s) and/reference(s), respectively, and where the determined efficacy coefficient(s) are further compared with each other to provide comparative transport and/or efficacy properties of the drug or chemical compound.

The method of any of the previous clauses, wherein the method further comprises composing a comparison criterion for an intended application of the drug or chemical composition from a set of variables, and the comparison criterion includes at least one time-invariant parameter.

6. A method for determining whether a drug or chemical compound is suitable for an intended purpose, the method comprising the steps of:
a) placing a defined amount of a drug or chemical compound specimen in a source compartment;
b) establishing a contact of the specimen with a transfer media which is able to transport the compound to at least one target compartment;
c) measuring the concentration of that the drug or chemical compound or its derivatives, or at least one efficacy coefficient, in at least one of the target compartments;
d) processing the measured data by time convolution procedure in real numbers without application of a preselected kinetic model;
e) calculation of time-invariant parameters comprising a set including at least a transport constant, coefficient of partition, kinetic parameter and optionally efficacy coefficient, from the processed data;
f) repeating steps c)-e) until desired time of the experiment is reached;
g) generate a model-free equation for the compound transport between the compartments;
h) calculate the non-dimensional Deborah rate number for the data from steps c)-g);
i) optionally calculating a comparison criterion between the specimens or with the reference or control specimen;
j) based on the calculated non-dimensional Deborah rate number in step h), provide a model-free time-invariant transport and/or efficacy properties of the drug or chemical compound; and
k) based on model-free time-invariant transport and/or efficacy properties determine whether the drug or compound is suitable for the intended purpose

7. The method of clause 6, wherein the drug or compound is a potential candidate for a lead design, lead optimization and/or clinical trial(s) and based on the determined model-free invariant transport and/or efficacy properties in step j), the drug or compound is included into or excluded from the lead design, lead optimization and/or clinical trial.

8. The method of clause 6 or 7, wherein the intended purpose is safe use of the tested drug or chemical by pregnant and/or breast-feeding women.

### Example 1—Simulation

Here the data simulating drug release are represented by the data set of Anscombe's quartet [8]. It comprises 4 data sets that have identical simple descriptive statistics but have very different distributions and appear different when graphed. Each Anscombe's dataset consists of 11 (x, y) points with the linear regression line y=3.00+0.50-x and the coefficient of determination 0.67.

For the purpose of the present invention, the first dataset [8] is used where x-values are assumed to be expressed in minutes and y-values are assumed to represent the relative concentration of the drug in the second (target) compartment Y₂, Table 1. The initial concentration in the first (source) compartment was adopted as equal to 13 units, and the volumetric ratio (ratio of volumes of distribution, VOD) of that compartment to the target compartment as 10:1 with the equilibrium partition coefficient equal to unity. The values of the concentration in the source compartment (balanced Y₁) are not initially known.

Using these starting points, the method of the present invention has been applied to these data, keeping only the conditions of the mass balance (no drug loss) and existence of the positive transport stimulus (Y₁>Y₂). This is not always the case in clinical practice, but acceptable here as all these data are from the simulation.

TABLE 1
Data for the Example 1 and computed values.

| | Given concentration data | | Calculated in the present invention | |
|---|---|---|---|---|
| | Y₂ | Y₁ | Y₁ | Y₂ |
| X (min) | (in target) | (in source) | (source) | (target) |
| 240 | 4.26 | 12.95 | 12.95 | 3.642 |
| 300 | 5.68 | 12.57 | 12.64 | 4.718 |
| 360 | 7.24 | 12.43 | 12.53 | 5.702 |
| 420 | 4.82 | 12.28 | 12.43 | 6.579 |
| 480 | 6.95 | 12.52 | 12.34 | 7.349 |
| 540 | 8.81 | 12.31 | 12.27 | 8.020 |
| 600 | 8.04 | 12.12 | 12.20 | 8.602 |
| 660 | 8.33 | 12.20 | 12.14 | 9.106 |
| 720 | 10.84 | 12.17 | 12.09 | 9.543 |
| 780 | 7.58 | 11.92 | 12.05 | 9.922 |
| 840 | 9.96 | 12.24 | 12.01 | 10.252 |

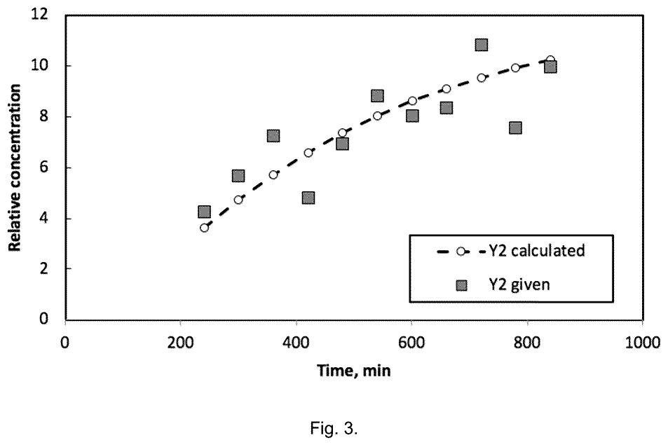

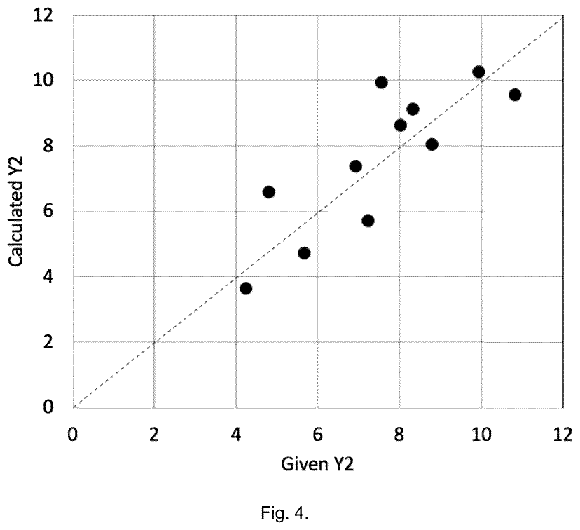

The calculations were made using time convolution process described above without any regression fit or any pre-selected pharmacokinetic model. The results are shown in FIG. 3 and FIG. 4 to compare given and calculated values, which show a very good agreement.

According to the present invention the extracted time-invariant parameters of this system are characteristic time T=47.6 min, non-dimensional transport constant K=25.3 and kinetic parameter [symbol] =1.70±0.03. It is possible that even better agreement could be obtained also with traditional regression models, but it is not possible to get these invariant parameters using common regression methods or other curve fitting procedures, as they generate numerical coefficients which do not usually have a physical meaning.

### Example 2—Clinical Case

Clinical data of Popovic et al. [6] have been used representing evaluation of diclofenac pharmacokinetics in a small number of healthy adults during a bioequivalence trial. In the spirit of the present invention, the data from [6] were taken as cumulative concentration of diclofenac, averaged for all 12 patients. Here the initial mass of the drug was known of 100 mg, and the instant volume of distribution (VOD-2) was directly quantifiable by diving this mass for the measured cumulative concentration analyzed (Table 2).

TABLE 2
Experimental data [6] processed with
the method of the present invention.

| | Tested c₂(t), | | | | Calculated c₂(t), |
|---|---|---|---|---|---|
| Time, h | mg/mL | VOD-2, L | [symbol] | Dr, ×10⁶ | mg/mL |
| 1.0 | 0.192 | 521.74 | — | — | 0.141 |
| 1.5 | 1.783 | 56.07 | 5.538 | 644.65 | 1.216 |
| 2.0 | 4.725 | 21.16 | 4.884 | 244.03 | 3.818 |
| 2.5 | 7.283 | 13.73 | 4.480 | 124.66 | 6.832 |
| 3 | 9.500 | 10.53 | 4.256 | 75.67 | 9.093 |
| 4 | 11.150 | 8.97 | 3.875 | 37.46 | 11.349 |
| 6 | 12.333 | 8.11 | 3.388 | 16.35 | 12.560 |
| 8 | 12.858 | 7.78 | 3.266 | 10.07 | 12.846 |
| 12 | 12.900 | 7.75 | 3.471 | 5.80 | 13.001 |
| 24 | 13.000 | 7.69 | 3.388 | 3.07 | 13.073 |

Calculations (3)-(5) have led to the following set of invariants for these experimental data: coefficient of partition of diclofenac L_{eq}=0.0834, transport constant K=11.894, and characteristic time T=0.338 h. Small L_{eq} value reflects the fact that diclofenac partition capacity is very favorable in these patients, and high K value reflects that diclofenac availability to the target is not very good at the beginning of the transport process (short times).

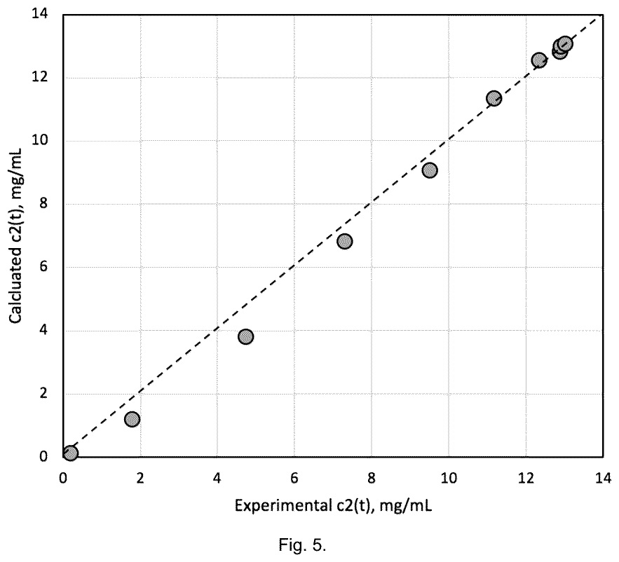

The kinetic parameter β>1 and is a time-dependent value in this case (Table 2), which is consistent with K and L_{eq} combinations: large [symbol] -values indicate a sigmoid-like drug transport release kinetics for t≥τ, initially retarded (high K) but then enhanced (low L_{eq}). The comparison of experimental and calculated data is shown in FIG. 5 indicating a very good assessment.

### Example 3—Hydrophilic Drug Release

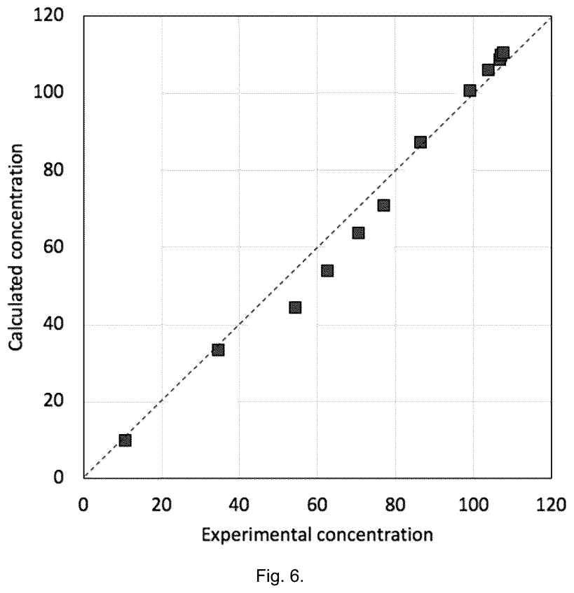

This example analyses experimental data for 12 h release of a hydrophilic matrix drug (Claritin D) as was disclosed in US patent application US2004081692 [9]. This cumulative drug release designated as ‘BT004’ (FIG. 3 in [9]) in that experiment was measured but the conditions were not clearly specified (no indications about initial dose, VOD neither partition coefficient). Hence for the purpose of the calculations the initial dose of 200 mg has been adopted, and the target compartment VOD was set to unity, as in the Example 1. For the method of the present invention, this was sufficient to make calculations as shown in Table 3 and FIG. 6.

TABLE 3
Hydrophilic drug BT004 release 12 h [9].

| Time min | Experimental c₂(t) | [symbol] (t) | Calculated c₂(t) |
|---|---|---|---|
| 1.5 | 10.55 | — | 9.819 |
| 10.5 | 34.483 | 0.649 | 33.370 |
| 19.8 | 54.295 | 0.722 | 44.381 |
| 30 | 62.528 | 0.751 | 53.930 |
| 45 | 70.399 | 0.749 | 63.835 |
| 60 | 77.059 | 0.745 | 70.969 |
| 120 | 86.248 | 0.702 | 87.319 |
| 240 | 99.046 | 0.655 | 100.565 |
| 360 | 103.777 | 0.636 | 106.094 |
| 480 | 106.61 | 0.624 | 108.772 |
| 600 | 107.089 | 0.613 | 109.999 |
| 720 | 107.651 | 0.600 | 110.339 |

For the invariant values, kinetic parameter β was seen slightly depending on time, but could be reasonably averaged to [symbol] =0.677±0.058. This indicates that Claritin D release in the experiment ‘BT004’ essentially follows a power-like law with almost no lag.

The obtained characteristic time is T=8.96 min meaning after 10 min of exposure the kinetic law of the release is stabilized. Transport constant K=4.294 and partition coefficient of 0.325 show that this drug is well distributed—better than e.g. diclofenac of Example 2.

### Example 4—Assessment of Floating Drug Delivery System

This example analyses data for nine different formations of the delivery of antihypertensive drug trandolapril as presented in the Australian patent application AU2021105584 [10]. All these formulations (marked F1 . . . F9) have 200 mg of trandolapril but varied amounts and presence of the supplements (tables total mass about 600 mg). The dissolution kinetics was assessed during 12 h in 500 mL (=VOD-2) of 0.1N HCl solution which imitates gastric fluid, with a paddle type apparatus. The floating time of the tablets has been also assessed there.

In the patent application release kinetics was estimated by fitting data to pre-selected models: a) linear zero order kinetics (zero power), b) first order rate kinetics by power of 1, c) Higuchi matrix by power of ½, and d) Hixson-Crowell erosion equation by power of ⅓. The patent application has selected the best composition for which the release kinetics is closer to the zero order (linear), and there the best choice was for the formulation F4 (found kinetic order n=0.603), however no more specific details were given in that application [10].

In this present invention, the tabulated data of the patent application were analyzed and the following invariant parameters (characteristic time τ, transport constant K, equilibrium coefficient of partition L_{eq}, kinetic parameter [symbol] ) have been obtained, Table 4.

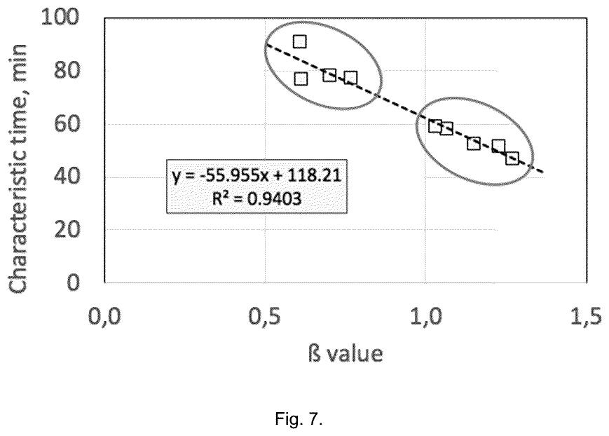

The analysis of the results according to the present invention has allowed discovery of the trends which were otherwise impossible to reveal with known methods. For instance, it was found that the formulations have the reverse linear trend between characteristic time τ and the kinetic parameter [symbol] (FIG. 7), splitting all formulations into two distinct clusters (the highest times and lowest [symbol] values are for formulations F2, F4, F5 and F7 respectively). These formulations have higher retention time and more flat release kinetics.

TABLE 4
Calculated values for trandolapril release from formulations F1 to F9.

| Case: | F1 | F2 | F3 | F4 | F5 | F6 | F7 | F8 | F9 |
|---|---|---|---|---|---|---|---|---|---|
| τ (min) | 58.36 | 78.32 | 59.03 | 77.46 | 91.05 | 52.49 | 76.82 | 51.77 | 47.12 |
| K | 11.80 | 5.79 | 6.44 | 2.33 | 2.34 | 2.89 | 2.60 | 2.57 | 5.45 |
| L_{eq} | 3.36 | 1.33 | 2.83 | 1.15 | 0.99 | 1.79 | 1.00 | 1.71 | 1.59 |
| [symbol] | 1.07 | 0.70 | 1.03 | 0.77 | 0.61 | 1.15 | 0.61 | 1.23 | 1.27 |

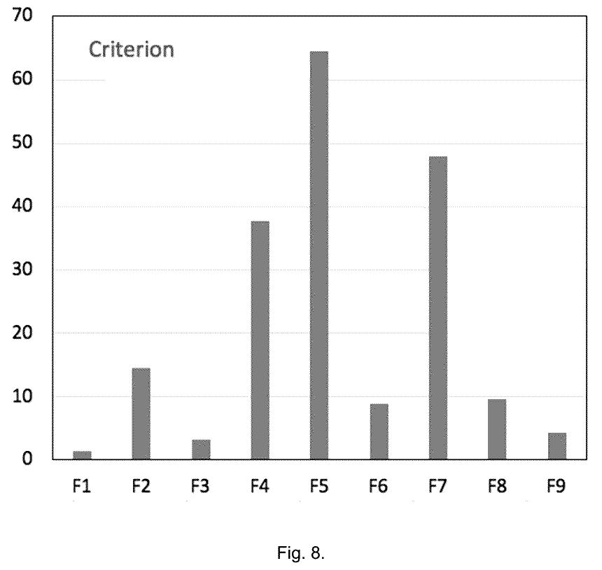

Hence for the target properties of the best drug formulation set up in the patent application but expressed in the terms of the present invention would be not a zero order kinetics requirement, but a composite criterion combining maximal characteristic time minimal transport constant K, partition coefficient L_{eq} and preferably [symbol] <1 as the algebraic product: Criterion=τ/(K·L_{eq}· [symbol] )→max, which should be a good guideline for selection of the optimal formulation in this example. This is shown in FIG. 8 where the winner is the formulation F5, followed by F7 and F4.

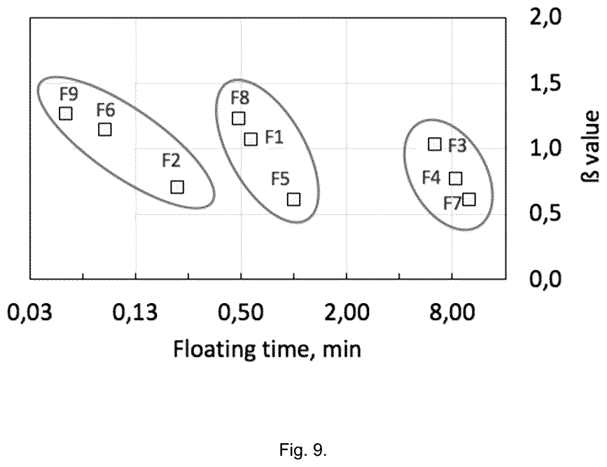

However, there are other limitations such as tablet floating time, which extension is expected to keep tablet longer in the stomach environment. When this time is plotted vs. 0-values, it is seen that there are three distinct clusters with low, average and large floating times, FIG. 9. Here is seen that F5 combination has only 1 min floating time which might be too short, so one should select either F7 or F4 options to obtain optimized drug composition for this case, as F3 composition has too low criterion in FIG. 8.

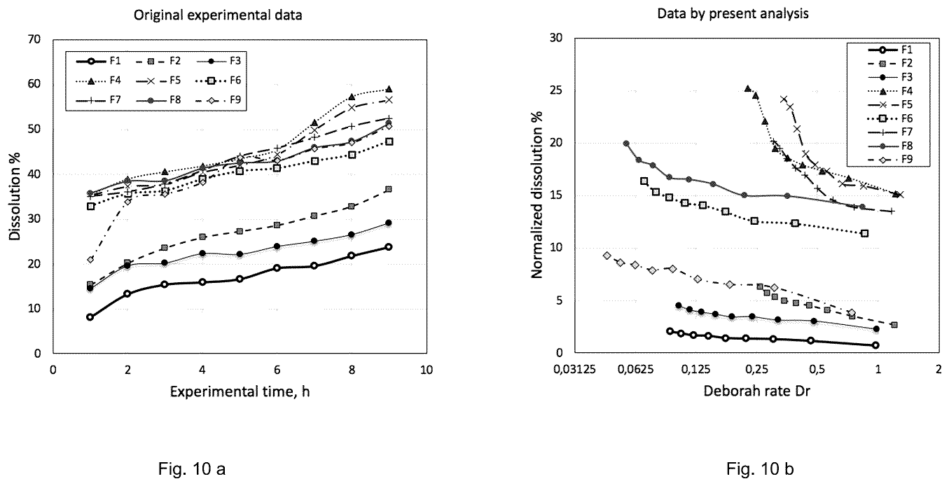

To demonstrate a visual benefit of the present analysis, the original data of the drug dissolution from are shown in FIG. 10a, and these data normalized by the transport constant of Table 4 are plotted vs. generalized Deborah rate (Dr) in FIG. 10b. One can see that the differences between these formulations F1-F9 are indeed remarkable when expressed via invariant values.

### Example 5—Gentamicin Release from the Coating

In this example the most detailed in vitro testing conditions have been used. Disk of diameter 15.5 mm and thickness of 2 mm made of medical titanium Grade 5 (Ti-6Al-4V) alloy were uniformly coated with a mixture of 1395 μg/disk gentamicin palmitate and 3745 μg/disk palmitic acid in triplicate (9 disks=3 disks×3 repetitions).

Disks were placed into a glass tube filled with 2 mL distilled water at constant temperature 37° C. and aliquots of 0.050 mL were periodically extracted without refilling the remining volume. The concentration of gentamicin in these samples was measured in mg/L and based on this, the amount of gentamicin in the remaining solution was calculated. The procedure was repeated until 336 h have been accumulated, and the average of three measurements has been calculated, Table 5.

After 336 hours of measurements, totally 0.5 mL of liquid is removed and about 60 μg of the drug is taken off the system. This represents of 25% of the total liquid volume and about 23.5% of the total drug released from the disk. With unknown solubility from the beginning, it is difficult to say how this drug removal affects the solubility rate, diffusion rate (concentration gradient uneven) and about the release kinetics in general (besides just making a curve fitting). As liquid is being periodically removed, the VOD-2 in the target compartment is getting smaller from 2 mL to 1.5 mL (being not constant) and thus the concentration of gentamicin there (c₂(t)) is affected too.

TABLE 5
Experimental data for the gentamicin elution from the coatings (one triplicate set is shown).

| | | | | | | | | Gentamicin removed (μg) | |
|---|---|---|---|---|---|---|---|---|---|
| | | Sample gentamicin amount, μg | | | | Remained in | | with aliquots | |
| Aliquots total, mL | Time, h | S1 | S2 | S3 | Average | the coating, μg | c₂(t), mg/L | instant | cumulative |
| 0 | 0 | 0 | 0 | 0 | 0 | 1395.0 | 0 | 0 | 0 |
| 0.05 | 1 | 183.8 | 180.5 | 197.2 | 187.2 | 1207.8 | 93.6 | 4.7 | 4.7 |
| 0.1 | 3 | 223.0 | 210.0 | 230.4 | 221.2 | 1173.8 | 113.4 | 5.7 | 10.3 |
| 0.15 | 6 | 167.4 | 167.4 | 180.9 | 171.9 | 1223.1 | 90.5 | 4.5 | 14.9 |
| 0.2 | 24 | 174.7 | 194.4 | 204.3 | 191.1 | 1203.9 | 103.3 | 5.2 | 20.0 |
| 0.25 | 48 | 183.6 | 190.8 | 210.9 | 195.1 | 1199.9 | 108.4 | 5.4 | 25.5 |
| 0.3 | 72 | 173.7 | 188.2 | 206.4 | 189.4 | 1205.6 | 108.3 | 5.4 | 30.9 |
| 0.35 | 96 | 182.9 | 207.1 | 209.6 | 199.9 | 1195.1 | 117.6 | 5.9 | 36.7 |
| 0.4 | 168 | 217.2 | 251.6 | 254.3 | 241.0 | 1154.0 | 146.1 | 7.3 | 44.1 |
| 0.45 | 264 | 226.4 | 261.2 | 254.8 | 247.5 | 1147.5 | 154.7 | 7.7 | 51.8 |
| 0.5 | 336 | 217.9 | 282.9 | 265.2 | 255.3 | 1139.7 | 164.7 | 8.2 | 60.0 |

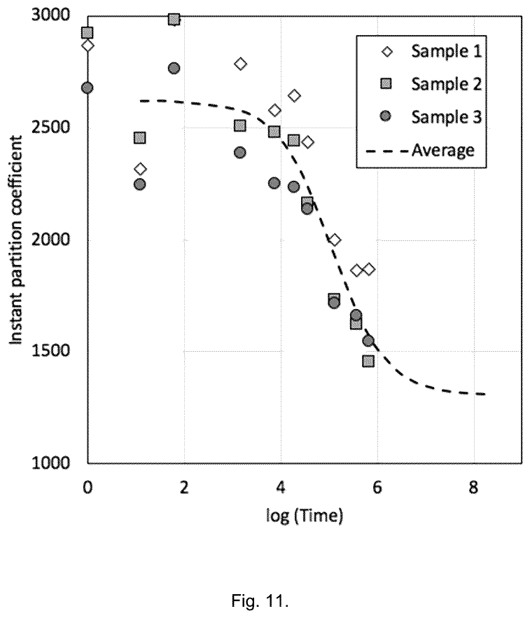

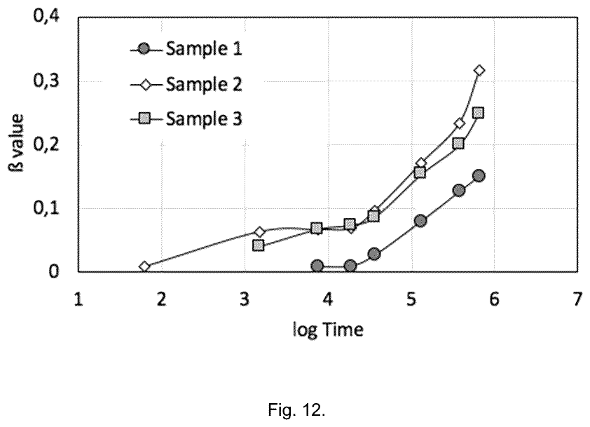

For the method of the present invention, exact VOD-1 for the source compartment has been first calculated. For the density of gentamicin palmitate of 1.5 g/cc and palmitic acid of 0.86 g/cc, taking into account the disk surface area of 188.69 mm², the expected dense coating thickness is about 28 μm at the beginning of the test, leading to the VOD-1 value (for the source compartment) of 5.285 mm³. Since VOD-2 is not constant due to aliquots takeoffs, the coefficient of partition is not constant either and will change with time, approaching an equilibrium value (L_{eq}). This coefficient for the data of Table 5 is shown in FIG. 11. For every sample in Table 5 assessed invariant values are shown in Table 6. For [symbol] -values, they were found not to be constant (FIG. 12) for the same reasons (forced VOD-2 change) as for partition coefficient, and hence they cannot in this experiment to be uniquely allocated to change of the elution mechanism.

However, as they stay well below unity (FIG. 12), this indicates that gentamicin palmitate release from these coatings is sufficiently slow and with almost no lag time. It is not possible to state that the kinetics of release is accelerating (due to increase of R values with time) because VOD-2 decreases and hence the capacity of the fluid in target compartment to take in additional drug becomes limited (some artificial increase of concentration).

TABLE 6
Extracted invariant values for samples 1-3 in Table 5.

| Sample No. | S1 | S2 | S3 |
|---|---|---|---|
| Transport constant K | 1376.26 | 1376.37 | 1165.78 |
| Characteristic time, h | 8.68 | 7.83 | 8.00 |

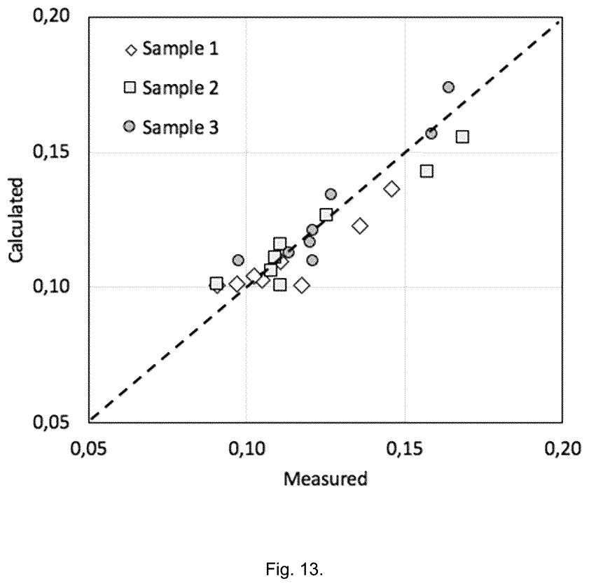

FIG. 13 shows the comparison of the measured and calculated concentrations of gentamicin. All these data have been obtained with the algorithm of the present invention and without assumption of the model. The method makes the use of the pre-selected models obsolete as the combination of invariants (τ, K, [symbol] , L_{eq}) is generally sufficient to understand the pharmacokinetics and in many cases to predict the drug behavior.

### Example 6—Efficacy Analysis for the Calcium Current Changes

In this example the data from publication by Oravecz et.al. was used, where authors have attempted to clarify the underlying mechanism of the limited inotropic action of selective sodium-calcium exchange (NCX) inhibition by a novel inhibitor molecule (ORM-10962) on canine ventricular myocytes. NCX is believed to be the main transport mechanism in regulation of cellular Ca²⁺ homeostasis in cardiac myocytes. The forward mode operation of NCX is considered as the main route of Ca²⁺ removal balancing the Ca²⁺influx generated by the L-type Ca²⁺ current (I_{Ca}) and the reverse mode NCX activity [11]. In these experiments 10 mM caffeine was applied at −80 mV under steady state conditions before and after equilibration with a novel inhibitor molecule ORM-10962. Standard delay of the caffeine administration after cessation of the stimulation protocol was ensured by using a software-controlled fast solution exchange [11]. The authors have observed in particular that under steady-state conditions I_{Ca} amplitude progressively decreased, but this reduction was significantly greater in the presence than in the absence of ORM-10962.

These experimental data have been analyzed in the present invention. The difference between I_{Ca} for control (0.1 μM ORM-10962) and test case were calculated and plotted vs. test time as shown in the publication [11]. In order to perform time convolution directly, the concentration of ORM-10962 should have been known in the target compartment, but here only the effect size (current measured) has been reported. Hence, an efficacy coefficient L_{Ca} linking the concentration and the current observed has been introduced, so the current differences are proportional to the active ORM-10962 concentration C₂(t): Δl_{Ca}=L_{Ca}·C₂(t). In this case the stimulus for the drug transport with the action (including all potentially important mechanisms) can be as S(t)=C₀−L_{eq}·C₂(t)=C₀−L_{eq}·Δl_{Ca}(t)/L_{Ca}. As in this experiment the drug has been directly administered, L_{eq}=1, and L_{Ca} is the efficacy parameter to be determined along with other invariants.

The results of the calculation according to the present invention have shown that this ORM-10962 action in respect to calcium current in myocytes can be expressed with the non-dimensional transport constant K=104.5, characteristic time 1.69 s and efficacy parameter L_{Ca}=42.123 nA/μM of the added drug. The kinetic parameter has the trend of a slight increase and can be approximated with a log trend [symbol] (t)=0.523·ln(t)−0.14, suggesting that drug action is somewhat accelerated after a small lag time.

The outcomes in this example have a direct meaning for comparison of a drug or a compound action with other drug candidates, controls or references. For instance, efficacy parameter L_{Ca} indicates how much Ca²⁺ current would change per every μM of administrated drug after a sufficiently long time. The transport constant K indicates how much the drug would affect the current at short time scales: the higher is the constant, the less change in the current would be seen.

Hence for one skilled in the art there would open a new possibility to screen various options between drug candidates by e.g., maximizing efficacy parameter (=more effect at longer times) with minimization of transport constant (=more effect at shorter times). Additionally, a user may set an extra criterion in larger or smaller characteristic time and kinetic parameter to express kinetic data vs. Deborah rate number (Dr) to fine tune the expected therapeutic outcome.

This example also shows that quantitative assessment of the drug efficacy might be possible without explicit knowledge of its pharmacokinetics, mode or mechanism of action or ADME features, because the invariants of the present invention are already implicitly including these (usually less known) external variables.

### Example 7—Unexpected Features Discovered in Caffeine Transport Tests

In this example data of caffeine transport measurements disclosed in publication are analyzed with a new method. There caffeine dose of initial concentration of 0.25 mg/mL was deployed in the source compartment simulating maternal side, and its concentration was also measured in the target compartment simulating fetal side, in the membrane-separated chip intended to mimic placental barrier [12]. The membrane had 0.4 micrometer pore size and was made of polyester track etched (PETE) insert, which was used bare (control tests) and with a double layer of cells (actual tests): human umbilical vein endothelial cells (HUVECs) and BeWo cells (derived from a human choriocarcinoma) to represent the endothelium in the fetal interface and the epithelium in the maternal interface [12]. The mean data used are from three independent experiments.

Authors have calculated the rate of caffeine transfer (% RT) for both maternal and fetal sides using the finite difference equation: % RT=ΔC_{f}/ΔCₘ·100%, where ΔC_{f} and ΔCₘ represent the change in caffeine concentrations in the fetal and maternal channels respectively during perfusion [12]. Initial and final caffeine concentrations from both the maternal and fetal sides were used when calculating the values for ΔC_{f} and ΔCₘ. To calculate the initial maternal and fetal caffeine concentrations, the values at a previous time point were used for both the actual and controlled experiments [12].

Whereas authors have observed a clear difference between caffeine concentrations in control and actual tests, the % RT data shown in do not have statistical significance (the p-value of the two-sided permutation t-test is 0.218). It is well known that the p-value is a common likelihood of observing the effect size, if the null hypothesis of zero difference is true, and commonly values p<0.05 are being considered ones having statistical significance. Authors did not report any other parameters about the caffeine transport in these conditions.

Using the method of the present invention, it was possible to analyze these data and extract the set of invariants (Table 7) as both concentrations in source (maternal side) and target (fetal side) compartment have been given explicitly (there is no need to estimate VOD for these two compartments). The kinetic parameter [symbol] was found to be time-dependent and being higher for actual tests ( [symbol] =3.2 . . . 5.4) than for control tests ( [symbol] =2.0 . . . 2.9).

TABLE 7
Extracted invariant values for control (no cells)
and actual (double cells layer) tests.

| | Characteristic | Transport | Coefficient of |
|---|---|---|---|
| Test | time τ, min | constant K | partition L_{eq} |
| Control | 40 | 162.77 | 5.61 |
| Actual | 27 | 6697.34 | 35.5 |

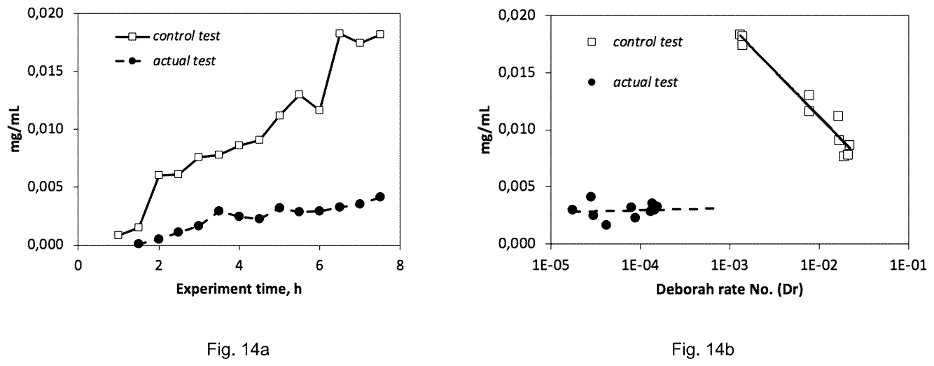

FIG. 14 shows the original data plotted vs. experimental time (a) and the same data (b) but plotted vs. generalized Deborah rate number (Dr) calculated in the present method. Here the following unexpected features of the caffeine transport in the system can be discovered:
in the control tests (with bare membrane only), caffeine concentration in the target compartment clearly increases with smaller Dr values, but it practically does not change for the actual tests (FIG. 14b) despite seen in the experiments directly.
characteristic time for actual tests is smaller than for control, which indicates ( [symbol] >1) that the lag time is shorter for cell-laden membrane even one could make opposite conclusion seeing original measurements only (FIG. 14a).
both K and L_{eq} values are significantly higher for actual tests than for control ones indicating that caffeine transport is significantly retarded by the cell barriers, but this retardation is not kinetical (=smaller characteristic time), as could be otherwise judged from the original data.
furthermore, the absence of changes of that concentration vs. Dr number for actual tests discovers the fact that cells are actively taking part in the transport process and do not readily allow caffeine to pass the barrier designed in the experiments [12], contradicting the statement in that caffeine is readily passing placental barrier.

The present method for the analysis of the experimental data of this example have shown unexpected features in the caffeine transport process, identified distinct differences in the invariants and suggested that the system used is likely requiring improvement if it is supposed to represent a placental barrier.

This example has also demonstrated benefits of application of generalized Deborah rate scale and combined assessment of the set of invariant values rather than some single numbers.

Additional Notes

The project leading to this patent application has received funding from the European Union's Horizon 2020 research and innovation under grant agreement No. 101036702.

Unlike prior art testing methods known to the inventors, the method of the preferred embodiments is internally consistent and directly related to known fundamental laws of physics and mathematics rather than empirical assumptions or pre-selected models. In use one thus relies of true experimental outcomes rather than artificial fitting of fragments of separate uncoupled values, being often away for relevant conditions.

The above detailed description together with accompanying drawings shows specific embodiments and examples in which the invention can be practiced. Such examples can include elements in addition to those shown or described. However, the inventors also contemplate examples using any combination or permutation of those elements shown or described (or one or more aspects thereof), either with respect to a particular example (or one or more aspects thereof), or with respect to other examples (or one or more aspects thereof) shown or described herein.

The above description is intended to be illustrative, and not restrictive. Also, in the above detailed description, various features may be grouped together to streamline the disclosure, whereas the inventive subject matter may consist in less than all features of a particular disclosed embodiment. Although the present invention has been described in more detail in connection with the above examples, it is to be understood that such detail is solely for that purpose and that variations can be made by those skilled in the art without departing from the spirit of the invention except as it may be limited by the following claims. Thus, the following claims are hereby incorporated into the detailed description, with each claim standing on its own as a separate embodiment, and it is contemplated that such embodiments can be combined with each other in various combinations or permutations.

In this document, the terms “a” or “an” are used, as is common in patent documents, to include one or more than one, independent of any other instances or usages of “at least one” or “one or more.” Also, in the following claims, the terms “including” and “comprising” are open-ended, that is, a system, device, article, or process that includes elements in addition to those listed after such a term in a claim are still deemed to fall within the scope of that claim.

Examples shown in the present invention foresee execution of computer instructions operable to configure and run an electronic device to perform these methods as described. An implementation of such instruction can be realized as a code, such as microcode, assembly language code, a higher-level language code, or user-independent executable code (like a computer program product), whether with or without a graphical user interface, stored or properly located on any computer-readable media during execution or at standby.

### LIST OF REFERENCES

[1] Rojas Gomez R., Valencia P. R. “In vitro-in vivo pharmacokinetic correlation model for quality assurance of antiretroviral drugs”. Colombia Médica (2015), 46: 109-116
[2] Gabrielsson J., Andersson R., Jirstrand M., Hjorth S. “Dose-Response-Time data analysis: an underexploited trinity”. Pharmacol. Reviews (2019), 71: 89-122.
[3] Gabrielsson J., Hjorth S. “Pattern recognition in pharmacodynamic data analysis”. The Amer. Assoc. Pharm. Scientists Journal (2016), 18: 64-91.
[4] Honek J. “Preclinical research in drug development”. Medical Writing (2017), 26: 5-8.
[5] Checkley S., MacCallum L., Yates J., Jasper P., Luo H., Tolsma J., Bendtsen C. “Bridging the gap between in vitro and in vivo: dose and schedule predictions for the ATR inhibitor AZD6738”. Sci. Reports (2015), 5: 13545.
[6] Popovic J. K., Atanackovic M. T., Pilipovic A. S., Rapaic M. R., Pilipovic S., Atanackovic T. M. “A new approach to the compartmental analysis in pharmacokinetics: fractional time evolution of diclofenac”. J. Pharmacokin. Pharmacodyn. (2010), 37: 119-134.
[7] Verotta D. “Fractional compartmental models and multi-term Mittag—Leffler response functions”. J. Pharmacokin. Pharmacodyn. 37 (2010), 209-215.
[8] Anscombe F. J. “Graphs in Statistical Analysis”. Amer. Statistician (1973) 27: 17-21.
[9] “Oral NNDs design using modeling based on fractional release times”, US patent application US2004/081692.
[10] “Fabrication and evaluation of gastro retentive tablet of trandolapril in treatment of essential hypertension”. Australian patent application AU021105584.
[11] Oravecz K., Kormos A., Gruber A., Marton Z., Kohajda Z., Mirzaei L., Jost N., Levijoki J., Pollesello P., Koskelainen T., Otsomaa L., Toth A., Papp J. G., Nanasi P. P., Antoons G., Varró A., Acsai K., Nagy N. “Inotropic effect of NCX inhibition depends on the relative activity of the reverse NCX assessed by a novel inhibitor ORM-10962 on canine ventricular myocytes”. Europ. J. Pharmacology (2018) 818: 278-286.
[12] Pemathilaka R. L., Caplin J. D., Aykar S. S., Montazami R., Hashemi N. N. “Placenta-on-a-Chip: in vitro study of caffeine transport across placental barrier using liquid chromatography—mass spectrometry”. Global Challenges (2019), 1800112.

What is claimed is:

1. A method for determining model-free time-invariant transport and/or efficacy properties of a chemical compound said method comprising the steps of:
a) placing a defined amount of a drug or chemical compound specimen in a source compartment;
b) establishing a contact of the specimen with a transfer media which is able to transport the compound to at least one target compartment;
c) measuring the concentration of the drug or chemical compound or its derivatives, or at least one efficacy coefficient, in at least one of the target compartments;
d) processing the measured data by time convolution procedure in real numbers without application of a preselected kinetic model;
e) calculation of time-invariant parameters comprising a set including at least a transport constant, coefficient of partition, kinetic parameter and optionally efficacy coefficient, from the processed data;
f) repeating steps c)-e) until desired time of the experiment is reached;
g) generating a model-free equation for the compound transport between the compartments;
h) calculating a non-dimensional Deborah rate number for the data from steps c)-g) representing the transport and/or efficacy properties; and
i) optionally calculating a comparison criterion between the specimens or with the reference or control specimen.

2. The method of claim 1, wherein data analysis is executed iteratively for discover unknown coefficient of partition of the compound between the compartments, and/or efficacy coefficient, and/or unknown volume of distribution.

3. The method of claim 1, wherein the data analysis and comparison between the experiments, control(s) and/reference(s) are being made versus non-dimensional Deborah rate number.

4. The method of claim 1, wherein the method comprising multiple experiments including control(s) and/reference(s), wherein at least one efficacy coefficient is also determined step c) for the multitude of experiments, control(s) and/reference(s), respectively, and where the determined efficacy coefficient(s) are further compared with each other to provide comparative transport and/or efficacy properties of the drug or chemical compound.

5. The method of claim 1, wherein the method further comprises composing a comparison criterion for an intended application of the drug or chemical composition from a set of variables, and the comparison criterion includes at least one time-invariant parameter.

6. A method for determining whether a drug or chemical compound is suitable for an intended purpose, the method comprising the steps of:
a) placing a defined amount of a drug or chemical compound specimen in a source compartment;
b) establishing a contact of the specimen with a transfer media which is able to transport the compound to at least one target compartment;
c) measuring the concentration of that the drug or chemical compound or its derivatives, or at least one efficacy coefficient, in at least one of the target compartments;
d) processing the measured data by time convolution procedure in real numbers without application of a preselected kinetic model;
e) calculating time-invariant parameters comprising a set including at least a transport constant, coefficient of partition, kinetic parameter and optionally efficacy coefficient, from the processed data;
f) repeating steps c)-e) until desired time of the experiment is reached;
g) generating a model-free equation for the compound transport between the compartments;
h) calculating the non-dimensional Deborah rate number for the data from steps c)-g);
i) optionally calculating a comparison criterion between the specimens or with the reference or control specimen;
j) based on the calculated non-dimensional Deborah rate number in step h), provide a model-free time-invariant transport and/or efficacy properties of the drug or chemical compound; and
k) based on model-free time-invariant transport and/or efficacy properties determine whether the drug or compound is suitable for the intended purpose.

7. The method of claim 6, wherein the drug or compound is a potential candidate for a lead design, lead optimization and/or clinical trial(s), and based on the determined model-free invariant transport and/or efficacy properties in step j), the drug or compound is included into or excluded from the lead design, lead optimization and/or clinical trial.

8. The method of claim 7, wherein the intended purpose is safe use of the tested drug or chemical by pregnant and/or breast-feeding women.
